(19) United States
(12) Patent Application Publication
(10) Pub. No.: US 2011/0105739 A1
(43) Pub. Date: May 5, 2011

(54) NOVEL MATRIX MATERIALS, METHODS FOR THE PRODUCTION THEREOF, AND USE THEREOF IN METHODS FOR ISOLATING BIOMOLECULES

(76) Inventors: Roland Fabis, Leverkusen (DE); Ralf Hammelreich, Langenfeld (DE)

(21) Appl. No.: 12/866,853

(22) PCT Filed: Feb. 20, 2009
(86) PCT No.: PCT/EP09/52048

§ 371 Date: Nov. 29, 2010

(30) Foreign Application Priority Data
Feb. 22, 2008 (DE) 10 2008 010 693.3

(51) Int. Cl. C07H 1/06; C07H 21/00; C08F 8/32; C08F 8/34; C08F 8/24; C08F 8/14; C08F 8/12

(52) U.S. Cl. 536/23.1; 536/25.4; 525/329.9; 525/329.8; 525/329.7; 525/329.4; 525/330.4; 525/330.5; 525/330.3; 525/350; 525/379; 525/384

(57) ABSTRACT

The invention relates to functionalized matrix materials or matrices that have structures of general formula (I) T-(C═O)O—B-D, methods for the production thereof, and the use thereof in methods for purifying and isolating biomolecules.

Novel matrix materials, methods for the production thereof, and use thereof in methods for isolating biomolecules.

The present invention relates to functionalized matrix materials or to matrices comprising these materials, which have structures of the general formula I

T—(C═O)O—B-D (I),
in which
T may be a polymer or copolymer comprising units of acrylic acid and/or derivatives thereof
B may be a covalent bond or an alkylene group having 1 to 6 carbon atoms
D may be a branched or unbranched C₂-C₂₀-alkyl radical or C₃-C₁₂-cycloalkyl radical, the carbon chain of which may optionally be interrupted, identically or differently, by one up to 10 oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s), or may be a group

—CH(XR¹)—CH₂(YR²)
in which
X and Y may each independently be oxygen, sulfur or —NR³— and
R¹ and R² may each independently be hydrogen or a branched or unbranched C₁-C₂₀-alkyl radical or C₃-C₁₂-cycloalkyl radical, the carbon chain of which may optionally be interrupted, identically or differently, by one up to 10 oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s),
R³ may be hydrogen or a C₁-C₆-alkyl radical which may be substituted by one or more hydroxyl group(s), thiol group(s) or amino group(s) and/or halogen atom(s),
R⁴ may be hydrogen or a C₁-C₆-alkyl radical which may be substituted by one or more hydroxyl group(s), thiol group(s) or amino group(s) and/or halogen atom(s),
   where, in the case that the support consists of a homopolymer of methacrylic acid, D must not be defined as —CH₂—CH₂OH.

Preference is given to structures of the general formula (I) in which
T may be a polymer or copolymer comprising units of acrylic acid and/or methacrylic acid and/or amides thereof and/or esters thereof
B may be a covalent bond or an alkylene group having 1 to 3 carbon atoms
D may be a branched or unbranched C₂-C₁₀-alkyl radical or C₃-C₁₀-cycloalkyl radical, the carbon chain of which may optionally be interrupted, identically or differently, by one up to three oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s), or may be a group

—CH(XR¹)—CH₂(YR²),
in which
X and Y may each independently be oxygen, sulfur or —NR³—,
R¹ and R² may each independently be hydrogen or a branched or unbranched C₁-C₁₀-alkyl radical or C₄-C₁₀-cycloalkyl radical, the carbon chain of which may optionally be interrupted, identically or differently, by one or with up to three oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s),
R³ may be hydrogen or a C₁-C₆-alkyl radical which may be substituted by one or more hydroxyl group(s), thiol group(s) or amino group(s) and/or halogen atom(s),
R⁴ may be hydrogen or a C₁-C₆-alkyl radical which may be substituted by one or more hydroxyl group(s), thiol group(s) or amino group(s) and/or halogen atom(s),
   where, in the case that the support consists of a homopolymer of methacrylic acid, D must not be defined as —CH₂—CH₂OH.

Particular preference is given to structures of the general formula (I) in which
T may be a polymer or copolymer comprising units of acrylic acid and/or methacrylic acid and/or esters thereof
B may be a covalent bond or a methylene group
D may be a branched or unbranched C₂-C₈-alkyl radical or C₃-C₈-cycloalkyl radical, the carbon chain of which may optionally be interrupted, identically or differently, by one up to two oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s), or may be a group

—CH(XR¹)—CH₂(YR²) in which
X and Y may each independently be oxygen, sulfur or —NR³—,
R¹ and R² may each independently be hydrogen or a branched or unbranched C₁-C₆-alkyl radical or C₄-C₆-cycloalkyl radical, the carbon chain of which may optionally be interrupted, identically or differently, by one or with up to two oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s),
R³ may be hydrogen or a C₁-C₃-alkyl radical which may be substituted by one or more hydroxyl group(s), thiol group(s) or amino group(s) and/or halogen atom(s),
R⁴ may be hydrogen or a C₁-C₃-alkyl radical which may be substituted by one or more hydroxyl group(s), thiol group(s) or amino group(s) and/or halogen atom(s),
   where, in the case that the support consists of a homopolymer of methacrylic acid, D must not be defined as —CH₂—CH₂OH.

Very particular preference is given to structures of the general formula (I) in which
T may be a crosslinked polymer consisting of units of methacrylic esters
B may be a covalent bond or a methylene group
D may be —CH₂—CH(OH)—CH₂OH, —CH₂—C(CH₂OH)₃, —CH₂—CH₂—O—CH₂—CH₂—OH
   or a group

—CH(XR¹)—CH₂(YR²)
in which
X and Y may each be oxygen,
R¹ and R² may each independently be hydrogen, —CH(OH)—CH₂OH, —CH₂—CH(OH)—CH₂OH, —CH₂—C(CH₂OH)₃, —CH₂—CH₂—O—CH₂—CH₂—OH.

Most preferred are structures of the general formula (I) in which
T may be an ethylene glycol dimethacrylate-crosslinked polymer consisting of units of preferably one or more methacrylic ester(s),
B may be a covalent bond or a methylene group,
D may be —CH₂—CH₂OH, —CH₂—CH(OH)—CH₂OH, —CH₂—C(CH₂—OH)₃, —CH₂—CH₂—O—CH₂—CH₂—OH
   or a group —CH(XR¹)—CH₂(YR²)
   in which
X and Y may be oxygen,
R¹ and R² may each independently be hydrogen, —CH(OH)—CH₂OH, —CH₂—CH(OH)—CH₂OH, —CH₂—C(CH₂OH)₃, —CH₂—CH₂—O—CH₂—CH₂—OH.

The present invention further relates to processes for preparing the compounds of the general formula (I) and to the use thereof in processes for purifying or for isolating biomolecules.

Biomolecules according to the present invention are understood quite generally to mean molecules which, as products of an evolutionary selection, by virtue of controlled and selective interaction, fulfill specific tasks for an organism and form the basis of its life function [cf. Römpp, Lexikon Biochemie and Molekularbiologie, Georg Thieme Verlag, Stuttgart 1999].

In particular, in the context of the present invention, nucleic acids, for example plasmid DNA, chromosomal DNA, total RNA, mRNA or RNA/DNA hybrids, are considered to be biomolecules.

The inventive use relates to the purification or isolation of these biomolecules from aqueous systems which comprise, for example, impurities such as fats or cell fragments inter alia.

In addition, the present invention relates to the isolation or purification of real or artificial biomolecules from reaction mixtures which result from laboratory or industrial synthetic conversions.

The invention relates especially to matrix materials or matrices which have the ability to reversibly immobilize nucleic acids under defined conditions. The invention relates additionally to processes for producing and to use of such matrix materials for producing magnetic matrices, and to the use thereof for purifying or isolating the desired biomolecules—especially nucleic acid(s).

The enormous scientific progress in the field of molecular biology—including the fields of gene technology and molecular diagnostics—has caused a high demand for rapid, reliable and automatable processes for isolating and purifying nucleic acids.

The biological samples which are used for techniques such as DNA identification or analysis may originate from a multitude of sources, for example animal and plant cells, feces, tissue and bone samples (biopsies) or blood. In addition, the samples may, however, also be taken from the soil, from foods, or from synthetic nucleic acid mixtures.

Many molecular biology processes, such as reverse transcription, cloning, restriction analysis, amplification and sequencing, necessarily require that the nucleic acids used in the particular processes are essentially free of impurities which can adversely affect the corresponding reaction processes or analysis methods. Such impurities generally include substances which catalyze or initiate the degradation or depolymerization of a nucleic acid, or substances which block the detection of the nucleic acid or mask the nucleic acid. Undesired impurities additionally include macromolecular substances, for example enzymes, proteins, polysaccharides or polynucleotides, and substances with a low molecular weight, such as lipids, enzyme inhibitors or oligonucleotides. Further impurities are dyes (pigments), trace elements (metals) or organic solvents.

These requirements have already led to establishment of a multitude of purification and isolation methods in the prior art over the years. For example, DNA can be obtained by salting out in the presence of phenol and trichloromethane. On the other hand, DNA and RNA can be obtained on mineral supports (for example silica) or—derivatized—silica resins, by the use of chaotropic salts. For use in automated processes, the use of magnetic particles—known as ‘magnetic beads’—in particular has become established.

First, Marko et al. [Analyt. Biochem. 121 (1982) 382] and Vogelstein et al. [Proc. Nat. Acad. Sci. 76 (1979) 615] recognized that, if the DNA in nucleic acid-containing extracts is exposed to high concentrations of sodium iodide or sodium perchlorate, only the DNA binds to the mechanically finely comminuted glass scintillation tubes and comminuted glass fiber membranes or glass fiber plates, while RNA and proteins do not bind. The DNA thus bound can be eluted with water in the simplest case. Later, numerous property rights based on this type of DNA isolation were accordingly published.

For instance, EP-A-389 063 relates to a method for isolating nucleic acids from a biological source. According to this method, the biological sources comprising nucleic acids, such as blood, cells, plasma, etc., are digested in high concentrations in the presence of chaotropic salts. Subsequently, the nucleic acids are bound to a silica surface. The nucleic acids are then washed and eluted.

The method disclosed in U.S. Pat. No. 5,155,018 describes the isolation of RNA from biological sources which comprise, as well as RNA, also DNA and other constituents. This involves acidifying the biological sample and mixing it with a chaotropic agent, for example a guanidinium salt. Silicate particles are added to the sample. Under the given conditions, RNA binds to the silicate particles.

Subsequently, in this method too, the RNA is removed from the particles.

Colpan et al. describe, in international patent application WO-A-95/01359, a method for purifying and separating nucleic acid mixtures by adsorbing the nucleic acid from an alcohol-containing solution with high ionic strength. The adsorption solution comprises, as well as alcohol in a concentration of 1 to 50% by volume, salts in a concentration of 1 to 10 M, preference being given to the chaotropic salts, for example guanidinium thiocyanate, sodium perchlorate or guanidinium hydrochloride.

WO-A-95/21849 further provides a method for separating double-strand and/or single-strand nucleic acids from sources which comprise these nucleic acids. In this process too, the nucleic acids are adsorbed on mineral supports under conditions which allow binding of the desired nucleic acid species, while the undesired nucleic acid species does not bind to this mineral support.

In order to bind predominantly single-strand nucleic acids to a mineral support and thus to separate them from double-strand nucleic acids, the treatment conditions for the two samples comprising both nucleic acid species are established appropriately with an aqueous mixture of salts, especially of chaotropic salts and alcohol. The unadsorbed double-strand nucleic acid can then be purified further or isolated by known methods.

Further methods are disclosed, for example, in European patent applications EP-A-512 767 and EP-A-515 484, in International patent applications WO-A-95/13368, WO-A-96/18731 and WO-A-97/10331, and in U.S. Pat. Nos. 4,923,978 and 5,057,426.

European patent application EP-A-707 077 describes the binding and the subsequent selective release of nucleic acid from a lysate by means of a water-soluble, weakly basic polymer which forms a precipitate with the nucleic acid under specific conditions. This precipitate can be removed from the aqueous solution, and the nucleic acid can be released employing high salt concentrations and under the action of strong bases, or by heating.

With regard to all above-described isolation or purification methods, it should additionally be pointed out that blood is a source for DNA analyses available in the greatest amounts, since blood samples are routinely taken for a wide variety of different reasons. Owing to the viscous properties and owing to the extremely high protein content of the composition, the automation of the isolation of nucleic acids from this starting material was a perpetual source of unexpected difficulties—a fact also demonstrated repeatedly in the prior art. In addition—with regard to automation—the handling of large sample volumes having relatively small amounts of DNA was found to be difficult.

It is thus an object of the present patent application to at least partly solve the problems known from the prior art which have been outlined above. The present invention is thus targeted, in the most general sense, at the demand for materials and methods which enable a rapid and efficient method for isolating nucleic acids from a mixture of the desired nucleic acids with impurities.

In addition, it is an object of the patent application to provide matrices or matrix materials which are reusable after use.

In one embodiment of the invention, this object is achieved by functionalized matrix materials of the general formula I and by methods for isolating one or more nucleic acids, for example plasmid DNA, chromosomal DNA, total RNA, m-RNA or RNA/DNA hybrids, for example, from biological samples which comprise impurities, such as proteins, fats, cell fragments, etc.

In a further embodiment of the invention, the present invention relates to a modified matrix of the general formula I and to processes for producing the matrix materials of the general formula I or matrices which have these matrix materials.

In a further aspect, the invention relates to a method for isolating biomolecules—especially nucleic acids—using the abovementioned matrix, which comprises the following steps:
(a) providing the inventive matrix;
(b) combining the matrix with a mixture comprising the biomolecule to be isolated or comprising preferably the nucleic acid to be isolated and at least one impurity;
(c) incubating the matrix and the mixture under conditions under which the biomolecule or preferably the desired nucleic acid is immobilized on the matrix;
(d) separating the matrix with the biomolecule or with the immobilized nucleic acid(s) from the mixture;
(e) combining the matrix with the biomolecule or with the immobilized nucleic acid(s) with an elution solution, which desorbs the desired biomolecule(s) or nucleic acid(s) from the matrix.

The synthesis of structures which correspond to the general formula I—proceeding from the corresponding epoxides—is well known from the prior art, since, for example, epoxy resins which contain partial epoxide or oxirane structures and which thus a suitable starting material for preparing compounds covered by the general formula I—constitute products which find use on the industrial scale (for example in the paints industry).

The corresponding inventive materials of the general formula I can be made readily available in a subsequent reaction step, by opening the epoxide ring (some examples of possible routes being via hydrolyis or alcoholysis).

In general, the inventive structures of the general formula I can be prepared from precursor molecules which have suitable precursor groups by numerous routes known from the prior art.

For example, suitable oxiranes can be prepared by the epoxidation (Prilezhaev reaction) of alkenes, or from hydroperoxides during the autoxidation of olefins. In addition, the epoxide partial structures desired in the intermediate can be obtained proceeding from suitable precursor groups by dehydrohalogenation of halohydrins or by the reaction of carbonyl compounds with sulfur ylide nucleophiles (Corey). For further explanation, it should be noted that such structures are also obtainable by the epoxidations of allyl alcohols (Sharpless-Katsuki epoxidation) or of olefins with sodium hypochlorite under manganese(III) catalysis (Jacobsen-Katsuki epoxidation).

In addition, the reaction of α-halo esters with carbonyl compounds in the presence of sodium ethoxide opens up a route to the corresponding structurally related 2-(ethoxycarbonyl)-oxiranes (Darzens reaction).

In the context of the present invention, it is, however, preferred also to incorporate epoxy-functionalized monomers into the polymer which forms the support, such that the polymerization already affords an intermediate which has the desired partial oxirane structure and which can be converted in the subsequent step with ring opening of the epoxide ring—for example via the route of acidic hydrolysis or of alcoholysis—to the desired end product of the general formula I.

A terpolymer based on acrylic acid derivatives, into which the epoxide partial structures by copolymerization of a monomer having a partial oxirane structure and optionally of a further monomer which serves as a crosslinker, preferably constitutes an advantageous intermediate.

More preferably, in the case of polymerization of methacrylates—preferably in the presence of glycol dimethacrylate which acts as a crosslinker—the epoxy-functionalized monomer which may be added is glycidyl methacrylate. The resulting polymer thus has the partial oxirane structures required for the preparation of the inventive compounds of the general formula I (X═O and R¹ together with R² and Y forms a single bond), which can be converted by the subsequent hydrolysis/alcoholysis—or via other conversions known from the prior art—easily to the target compounds of the general formula I.

Such a functionalized polymer based on methacrylic acid derivatives—especially methacrylic esters and glycidyl methacrylates—is a particularly preferred intermediate in accordance with the invention.

Matrix materials of the general formula I may additionally be prepared by simple transesterification reactions—preferably of acrylic or methacrylic esters which have a short-chain partial alcohol structure. Examples of such reactants include the esters of acrylic acid or methacrylic acid with C₁-C₃-alcohols.

The geometric form in which the polymer(s) functionalized in accordance with the invention or the matrix materials is/are present is to a very substantial degree unimportant here. For example, the surfaces may be in the form of particles, membranes, nonwovens or wovens.

The matrix may also be present as a composite in which the matrix material is present, for example, in combination with an inorganic polymer—for example silica (SiO₂)—or applied to a metallic surface—for example gold or iron—or combined therewith in any other form.

Accordingly, the term “matrix” in the context of the invention should be understood as a matrix material when the latter is present in singular form. However, matrix also refers to the combination of the inventive matrix materials with other components, for example the abovementioned composites.

It is quite generally possible, for the production of the matrix, to apply the functionalized polymers (matrix materials) to any hydrophilic or hydrophobic—optionally precoated—surface, which results in a broad spectrum of application (including in multiwell systems, for example microtiter plates or microfluidic systems).

In the context of the invention, the support (T) of the matrix material or of the matrix is formed by a polymer, the term polymer being understood in its broadest definition. According to the invention, polymers in the context of the present invention represent addition polymers, polycondensates or polyadducts. According to the present invention, the support may be formed from homopolymers, copolymers, terpolymers, etc., or from mixtures of different polymeric constituents. The identical or different polymers may—if desired—be crosslinked with one another using a crosslinker. The term “support” in the context of the invention means the support of the functional group —(C═O)O—B-D.

To produce the matrix material, suitable monomers which have at least one partial structure, which are suitable for polymerization (or polycondensation or formation of polymeric adducts) and which have a second partial structure which has a precursor function to form the partial structure —(C═O)O—B-D. In the case of suitable groups (B and D) in the starting material, it may be possible to dispense with the detour via a precursor. This may be the case, for example, when this moiety does not/these moieties do not adversely affect the polymerization, or the later function thereof is not impaired under the reaction conditions of the polymerization.

According to the invention, the abovementioned substituents—unless stated otherwise—are defined as follows in the context of the present invention:

C₁-C₆-alkyl—also referred to generally merely as “alkyl”—is generally a monovalent, branched or unbranched hydrocarbon radical which has 1 to 6 carbon atom(s) and may optionally be substituted by one or more halogen atom(s)—for example fluorine—or one or more hydroxyl group(s), which may be the same as or different than one another. Examples include the following unsubstituted hydrocarbon radicals:

methyl, ethyl, propyl, 1-methylethyl(iso-propyl), butyl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl.

The same applies to other—for example smaller or larger—alkyl radicals, for example C₁-C₃-alkyl or C₄-C₆-alkyl.

“Higher alkyl radical” is a monovalent branched or unbranched C₇-C₂₀-alkyl radical which may optionally be substituted by one or more halogen atom(s)—preferably fluorine—which may be the same as or different than one another. Examples include the following, preferably unsubstituted, hydrocarbon radicals: branched or unbranched heptyl, octyl, nonyl, decyl, undecyl, dodecyl, tetradecyl, hexadecyl, dodecadecyl and eicosyl.

Alkylene is generally an unbranched divalent hydrocarbon radical of 1 to 6 carbon atoms or a branched divalent hydrocarbon radical of 3 to 6 carbon atoms, for example methylene, ethylene, propylene, 2-methylpropylene, pentylene and the like.

Cycloalkyl is—unless defined otherwise—a saturated monovalent cyclic hydrocarbon radical which has 3 to 8 carbon atoms and which may optionally be substituted by one or more halogen atom(s)—preferably fluorine—or by one or more hydroxyl group(s) which may be the same as or different than one another. Examples include: cyclopropyl, cyclohexyl, cycloheptyl or cyclooctyl. This carbon chain may be interrupted by one or more heteroatoms—for example nitrogen, oxygen or sulfur. Examples include 1,4-dioxixane (dioxane), 2,5-dihydrofuran, tetrahydrofuran, γ-pyran, 2H-1,3-dioxole, tetrahydropyrrole, 2,5-dihydropyrrole, piperidine, piperazine, tetrahydrothiophene, morpholine, 1,2-oxathiolane, 1,3-thiazole, 1H-4,1,2-thiadiazine, 1,2-oxathiepane, γ-thiopyran, 1,4-thiazixine(1,4-thiazine).

Preferred examples of oxygen-containing, heterocyclic and hydroxyl-substituted cyclic systems include, for example, the carbohydrates (pentoses and also hexoses), for example: arabinoses, xyloses or riboses, or glucoses, mannoses, galactoses, fructoses or sorboses.

Halogen is—unless stated otherwise—fluorine, chlorine, bromine or iodine, but preferably fluorine and chlorine.

In the context of the present invention, “support material” means the polymeric base structure which is formed by the polymerization of the starting monomers, optionally with a crosslinker and optionally other assistants. According to the invention, assistants are understood to mean primarily substances which influence, for example, the chemical or physical properties of the polymer. These include, for example, pore formers which influence the porosity of the polymer, or substances which impart magnetic properties to the polymer. The support material may—as desired—be bonded to a material which has a permanent or temporary magnetic moment.

The present invention further relates to a process for purifying or isolating nucleic acids, comprising: the provision of the inventive modified matrix which is to be used in the process. The subsequent immobilization step consists in combining the matrix with the mixture which comprises the nucleic acid to be isolated and at least one impurity under conditions under which the desired nucleic acid is adsorbed on the matrix, separating the matrix with the immobilized nucleic acid from the mixture and desorbing the nucleic acid, which desorbs the desired nucleic acid(s) from the matrix.

The exact reaction conditions which are necessary to ensure adsorption and desorption of the nucleic acids on and from the matrix depend on various factors. These include essentially the properties of the matrix, the nature of the desired nucleic acid (DNA or RNA, molecular weight and nucleotide sequence composition, pK_{b} and pKₐ of the basic and any acidic partial structures present, the density of these structures on the surface, and not least the ability of the matrix to bind (to) the nucleic acid(s)).

In addition, it is not ruled out that impurities in the mixture interfere with the binding step in a disadvantageous manner in the course of adsorption of the nucleic acid(s).

The mixtures used here, which comprise the desired nucleic acids, may firstly originate from synthetic reaction mixtures—as obtained, for example, in PCR—or—as already mentioned—from biological samples.

The biological samples comprising nucleic acid(s) are understood—in the context of the present invention—to mean cell-free sample material, plasma, body fluids, for example blood, serum, cells, leucocyte fractions, crusta phlogistica, sputum, urine, sperm, feces, smears, puncture fluids, tissue samples of any kind—for example biopsies, tissue parts and organs, food samples which contain free or bound nucleic acids or nucleic acid-containing cells, environmental samples which comprise free or bound nucleic acids or nucleic acid-containing cells—for example organisms (uni- or multicellular; insects, etc.), plants and plant parts, bacteria, viruses, yeasts and other fungi, other eukaryotes and prokaryotes, etc.

In the case of use of such samples, it is, however, first necessary to destroy the cells in order to make the nucleic acids accessible to the adsorption step at all.

When the desired nucleic acid is RNA, the adsorption conditions have to be adjusted such that the adsorption of the desired RNA is promoted. When DNA is also present as well as RNA in the mixture, the adsorption conditions can be adjusted such that the binding of one species—in this case especially RNA—to the matrix is promoted preferentially. The specific adsorption conditions which, however, are required to enable the adsorption and ultimately the desorption of RNA depend on the properties of the matrix and have to be determined for each different type of a matrix.

The matrix used in accordance with the invention is preferably in a form which can be separated by the action of an external force from the mixture comprising the desired nucleic acid and other substances/impurities, once the mixture has been mixed with the matrix. It is understandable to the person skilled in the art that the type of external force which is suitable for the separation of the matrix from the mixture depends on the form and on the physical properties of the matrix. For example, in the simplest case, the separation can be effected under the action of gravity when the matrix is, for example, in the form of the solid phase of a chromatographic separation system.

On the other hand, the matrix material can be added—optionally batchwise—to the mixture of the nucleic acid and impurity/impurities, and then removed again by decanting or filtering.

One external force which can be employed in the isolation process according to the invention may be a liquid which is under a high pressure, in which case the matrix forms the stationary phase of high-pressure liquid chromatography.

Other forms of external forces which are suitable for use in the process proposed in accordance with the invention include vacuum filtration, centrifugation or preferably magnetic force.

In the case of use of magnetic force, useful matrix materials or matrices are preferably those which comprise a permanently or temporarily magnetic material. In the context of the present invention, preference is given to using ferro- or ferrimagnetic particles, preferably selected from the group consisting of: γ-Fe₂O₃ (maghemite), Cr₂O₃, and ferrites, especially of the (M²⁺O)Fe₂O₃ type where M²⁺ is a divalent transition metal cation and preferably Fe₃O₄ (magnetite). However, other ferro- or ferrimagnetic particles may likewise be used. These particles have a mean particle diameter of less than 5 μm, preferably of less than 1 μm, and more preferably within a range between 0.05 and 0.8 μm, and most preferably within a range from 0.1 to 0.4 μm.

Examples of suitable, commercially available ferro- or ferrimagnetic particles are magnetic particles based on γ-Fe₂O₃—such as Bayoxide® E AB 21, based on ferrimagnetic magnetite as the Bayoxide® E 8706, E 8707, E 8710 and E 8713H type (obtainable from Lanxess AG, Leverkusen, Germany), and also as Magnetic pigment 340 and as Magnetic pigment 345 (obtainable from BASF AG, Ludwigshafen am Rhein, Germany).

In addition, it is also possible to use superparamagnetic materials. Useful superparamagnetic materials include Fe, Fe₃O₄, Fe₂O₃, superparamagnetic ferrites, Co, Ni, and binary and/or ternary compounds (alloys).

The examples here include iron oxide crystals having a diameter of 300 angstrom or less.

The examples which follow are intended to illustrate the invention without restricting it.

1. Production of Modified Beads with a Polymethacrylate Surface with Functional Groups of the General Formula I

[D=CH(XR¹)—CH₂(YR²) where X and Y═O; R¹ and R²═H (1.1) or —CH₂—C(CH₂OH)₃ (1.2)]:

1.1 Hydrolysis of the Epoxide

5 g of epoxide-functionalized magnetic polymer prepared by polymerizing methyl methacrylate using ethylene glycol dimethacrylate as the crosslinker and glycidyl methacrylate as the support of the epoxide functionality, are suspended in a 250 ml flask in 100 ml of a 100 mM aqueous hydrochloric acid solution. The reaction mixture is degassed twice using a water-jet vacuum (rotary evaporator) and stirred at a temperature of 60° C. over a period of approx. 12 h. The reaction mixture is transferred to a suction filter, and the modified magnetic polymer particles are washed twice each with deionized water, twice with 10 mM hydrochloric acid, twice with 100 mM aqueous sodium phosphate solution (pH=6) and finally twice with deionized water, and then resuspended in 100 ml of deionized water.

1.2 Alcoholysis of the Epoxide

5 g of epoxide-functionalized magnetic polymers prepared by polymerization of methyl methacrylate using ethylene glycol dimethacrylate as a crosslinker and glycidyl methacrylate as the support of the epoxide functionality are suspended in a 250 ml flask in 100 ml of a 100 mM aqueous hydrochloric acid solution. Then 2.5 g of pentaerythritol are added to the suspension. The reaction mixture is degassed twice under water-jet vacuum (rotary evaporator), and then stirred at a temperature of 70° C. over a period of approx. 12 h. The reaction mixture is transferred to a suction filter, and the modified magnetic polymer particles are washed four times with deionized water and then resuspended in 100 ml of deionized water.

2. Production of Modified Beads with a Polymethacrylate Surface with Functional Groups of the General Formula I

[D=—CH₂—CH(OH)—CH₂(OH) (2.1) or —CH₂—C(CH₂OH)₃ (2.2)]:

2.1 Transesterification of the Methyl Methacrylate with Glycerol

15 ml of a suspension of a magnetic polymer consisting of methyl methacrylate and ethylene glycol dimethacrylate or divinylbenzene in ethanol are introduced into a glass suction filter of porosity 4 and washed four times with anhydrous diglyme or toluene. The residue is taken up in a mixture of 5 g of glycerol in 45 ml of diglyme or toluene, and the mixture is transferred into a 100 ml three-neck flask. Subsequently, the flask is provided with a reflux condenser and the suspension is degassed twice. While stirring (100 rpm), the reaction mixture is subjected to transesterification at a reaction temperature of 120° C. over a period of 16 hours. Thereafter, the magnetic polymers are washed four times with demineralized water and three times with ethanol, and stored under ethanol.

2.2 Transesterification of the Methyl Methacrylate with Pentaerythritol

15 ml of a suspension of a magnetic polymer consisting of methyl methacrylate and ethylene glycol dimethacrylate or divinylbenzene in ethanol are introduced into a glass suction filter of porosity 4 and washed four times with anhydrous diglyme or toluene. The residue is taken up in a mixture of 5 g of pentaerythritol in 45 ml of diglyme or toluene, and the mixture is transferred into a 100 ml three-neck flask. Subsequently, the flask is provided with a reflux condenser and the suspension is degassed twice. While stirring (100 rpm), the reaction mixture is subjected to transesterification at a reaction temperature of 120° C. over a period of 16 hours. Thereafter, the magnetic polymers are washed four times with demineralized water and three times with ethanol, and stored under ethanol.

3. Purification of DNA with Diol-Modified (Hydrolysis) Magnetic Polymers

Using each of the four buffer systems listed below, two DNA samples were purified. The average yield was determined. The magnetic particles used were diol-modified particles (see 1.1!).

a) 190 μl of RLT buffer (QIAGEN) [guanidinium thiocyanate-containing buffer (pH 6.7-7.2)] and 225 μl of ethanol

b) 230 μl of P1/P2/P3 buffer (QIAGEN) and 175 μl of ethanol [buffer P1: chelating agent-containing buffer in the range from pH 7.8 to 8.2; buffer P2: a detergent-containing alkaline lysis buffer (pH 13); buffer P3: acetate-containing buffer (pH 5.4 to 5.6)

c) 190 μl of RLT buffer (QIAGEN) and 100 μl of diglyme

d) 190 μl of RLT buffer (QIAGEN) and 100 μl of triglyme

e) 190 μl of RLT buffer (QIAGEN) and 100 μl of 1,4-dioxane

After the addition of 20 μl of bead suspension (corresponding to 49 mg of beads per ml), the mixture was incubated at 1100 rpm using a thermo-shaker over a period of 2 min. The reaction mixture was separated using magnetic force and the supernatant was discarded. Thereafter, 400 μl of wash buffer PE (QIAGEN) [TRIS-containing buffer (pH 7.9-8.1)] were added and the reaction mixture was incubated at 1100 rpm using a thermo-shaker over a period of 1 min. The reaction mixture was then separated using magnetic force, and the supernatant was removed. This wash step was repeated. After the removal of the supernatant, the reaction vessels were dried over a period of 10 min, in order to remove residual ethanol.

Thereafter, 300 μl of RNase-free water were added. The resulting suspension was mixed by means of a thermo-shaker over a period of one minute. The suspension was separated again using magnetic force, and the supernatant was discarded. Subsequently 100 μl of the aqueous solution of 100 mM TRIS buffer (pH=9.5) were added. Thereafter, the suspension was mixed at 1100 rpm by means of a thermo-shaker over a period of 1 min. The elution was conducted using magnetic force and a magnetic separating apparatus. Finally, the elution step was repeated and the DNA content was determined by an OD determination and by gel electrophoresis to obtain the following results:

| Buffer system | RSD 001 [μg DNA] | RSD 002 [μg DNA] |
|---|---|---|
| A | 0.98 | 0.82 |
| B | 1.36 | 1.54 |
| C | 2.44 | 3.62 |
| D | 4.02 | 8.71 |
| E | 1.45 | 1.50 |

The present invention thus relates to matrix materials of the general formula I

T-(C═O)O—B-D (I)
in which
T may be a polymer or copolymer comprising units of acrylic acid and/or derivatives thereof
B may be a covalent bond or an alkylene group having 1 to 6 carbon atoms
D may be a branched or unbranched C₂-C₂₀-alkyl radical or C₃-C₁₂-cycloalkyl radical, the carbon chain of which may optionally be interrupted, identically or differently, by one up to 10 oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s), or may be a group

—CH(XR¹)—CH₂(YR²)
in which
X and Y may each independently be oxygen, sulfur or —NR³—
and
R² and R² may each independently be hydrogen or a branched or unbranched C₁-C₂₀-alkyl radical or C₃-C₁₂-cycloalkyl radical, the carbon chain of which may optionally be interrupted, identically or differently, by one up to 10 oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s),
R³ may be hydrogen or a C₁-C₆-alkyl radical which may be substituted by one or more hydroxyl group(s), thiol group(s) or amino group(s) and/or halogen atom(s),
R⁴ may be hydrogen or a C₁-C₆-alkyl radical which may be substituted by one or more hydroxyl group(s), thiol group(s) or amino group(s) and/or halogen atom(s),

where, in the case that the support consists of a homopolymer of methacrylic acid, D must not be defined as —CH₂—CH₂OH.

The present invention relates more particularly to matrix materials of the general formula I

T-(C═O)O—B-D (I)
in which
T may be a polymer or copolymer comprising units of acrylic acid and/or methacrylic acid and/or amides thereof and/or esters thereof
B may be a covalent bond or an alkylene group having 1 to 3 carbon atoms
D may be a branched or unbranched C₂-C₁₀-alkyl radical or C₃-C₁₀-cycloalkyl radical, the carbon chain of which may optionally be interrupted, identically or differently, by one up to three oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s), or may be a group

—CH(XR²)—CH₂(YR²)
in which
X and Y may each independently be oxygen, sulfur or —NR³—,
R² and R² may each independently be hydrogen or a branched or unbranched C₁-C₁₀-alkyl radical or C₄-C₁₀-cycloalkyl radical, the carbon chain of which may optionally be interrupted, identically or differently, by one or with up to three oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s),
R³ may be hydrogen or a C₁-C₆-alkyl radical which may be substituted by one or more hydroxyl group(s), thiol group(s) or amino group(s) and/or halogen atom(s),
R⁴ may be hydrogen or a C₁-C₆-alkyl radical which may be substituted by one or more hydroxyl group(s), thiol group(s) or amino group(s) and/or halogen atom(s),

where, in the case that the support consists of a homopolymer of methacrylic acid, D must not be defined as —CH₂—CH₂OH.

The present invention preferably relates to matrix materials of the general formula I

T-(C═O)O—B-D (I)
in which
T may be a polymer or copolymer comprising units of acrylic acid and/or methacrylic acid and/or esters thereof
B may be a covalent bond or a methylene group
D may be a branched or unbranched C₂-C₈-alkyl radical or C₃-C₈-cycloalkyl radical, the carbon chain of which may optionally be interrupted, identically or differently, by one up to two oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s), or may be a group

—CH(XR¹)—CH₂(YR²)
in which
X and Y may each independently be oxygen, sulfur or —NR³—,
R¹ and R² may each independently be hydrogen or a branched or unbranched C₁-C₆-alkyl radical or C₄-C₆-cycloalkyl radical, the carbon chain of which may optionally be interrupted, identically or differently, by one or with up to two oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s),
R³ may be hydrogen or a C₁-C₃-alkyl radical which may be substituted by one or more hydroxyl group(s), thiol group(s) or amino group(s) and/or halogen atom(s),
R⁴ may be hydrogen or a C₁-C₃-alkyl radical which may be substituted by one or more hydroxyl group(s), thiol group(s) or amino group(s) and/or halogen atom(s),

where, in the case that the support consists of a homopolymer of methacrylic acid, D must not be defined as —CH₂—CH₂OH.

The present invention more preferably relates to matrix materials of the general formula I

T-(C═O)O—B-D (I)
in which
T may be a crosslinked polymer consisting of units of methacrylic esters
B may be a covalent bond or a methylene group
D may be —CH₂—CH(OH)—CH₂OH, —CH₂—C(CH₂OH)₃, —CH₂—CH₂—O—CH₂—CH₂—OH
   or a group

CH(XR¹)—CH₂(YR²)
in which
X and Y may be oxygen,
R¹ and R² may each independently be hydrogen, —CH(OH)—CH₂OH, —CH₂—CH(OH)—CH₂OH, —CH₂—C(CH₂OH)₃, —CH₂—CH₂—O—CH₂—CH₂—OH.

The present invention most preferably relates to matrix materials of the general formula I

T-(C═O)O—B-D (I)
in which
T may be an ethylene glycol dimethacrylate-crosslinked polymer consisting of units of preferably one or more methacrylic ester(s),
B may be a covalent bond or a methylene group,
D may be —CH₂—CH₂OH, —CH₂—CH(OH)—CH₂OH, —CH₂—C(CH₂—OH)₃, —CH₂—CH₂—O—CH₂—CH₂—OH
   or a group —CH (XR¹)—CH₂(YR²)
   in which
   X and Y may be oxygen,
   R¹ and R² may each independently be hydrogen, —CH(OH)—CH₂OH, —CH₂—CH(OH)—CH₂OH, —CH₂—C(CH₂OH)₃, —CH₂—CH₂—O—CH₂—CH₂—OH.

The present invention relates especially to matrices which comprise one of the above-described matrix materials, which is bonded to a material which has a permanent or temporary magnetic moment.

The present invention preferably relates to the above-described matrices which comprise, as materials having a permanent or temporary magnetic moment, which are ferri- or ferromagnetic and para- or superparamagnetic.

The present invention preferably relates to the above-described matrices which comprise a matrix material in the form of beads.

The present invention additionally preferably relates to the above-described matrices which comprise a matrix material in which the matrix is in the form of a composite.

The present invention additionally relates to a process for producing the above-described matrix material, which has the following steps:
a) the monomers which build up the support material and which bear the epoxide partial structure are polymerized, optionally in the presence of a material which possesses a permanent or temporary magnetic moment, and

b) the resulting polymers are subjected to a hydrolysis, alcoholysis, aminolysis, or are reacted with a thiol, and
c) the derivatized polymers are isolated.

The present invention additionally relates to a process for producing the above-described matrix materials, which has the following steps:
a) the monomers which build up the support material and which bear the ester partial structure are polymerized, optionally in the presence of a crosslinker and/or of a material which possesses a permanent or temporary magnetic moment, and
b) the resulting polymers are subjected to a transesterification with an alcohol and
c) the derivatized polymers are isolated.

The present invention additionally relates to the use of matrix materials of the general formula I

T—(C═O)O—B-D (I),
in which
T may be a polymer or copolymer comprising units of acrylic acid and/or derivatives thereof
B may be a covalent bond or an alkylene group having 1 to 6 carbon atoms
D may be a branched or unbranched C₂-C₂₀-alkyl radical or C₃-C₁₂-cycloalkyl radical, the carbon chain of which may optionally be interrupted, identically or differently, by one up to 10 oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s), or may be a group

—CH(XR¹)—CH₂(YR²)
in which
X and Y may each independently be oxygen, sulfur or —NR³—
and
R¹ and R² may each independently be hydrogen or a branched or unbranched C₁-C₂₀-alkyl radical or C₃-C₁₂-cycloalkyl radical, the carbon chain of which may optionally be interrupted, identically or differently, by one up to 10 oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s),
R³ may be hydrogen or a C₁-C₆-alkyl radical which may be substituted by one or more hydroxyl group(s), thiol group(s) or amino group(s) and/or halogen atom(s),
R⁴ may be hydrogen or a C₁-C₆-alkyl radical which may be substituted by one or more hydroxyl group(s), thiol group(s) or amino group(s) and/or halogen atom(s),
for purifying or for isolating biomolecules.

The present invention preferably relates to the use of matrix materials of the general formula I

T—(C═O)O—B-D (I)
in which
T may be a polymer or copolymer comprising units of acrylic acid and/or methacrylic acid and/or amides thereof and/or esters thereof
B may be a covalent bond or an alkylene group having 1 to 3 carbon atoms
D may be a branched or unbranched C₂-C₁₀-alkyl radical or C₃-C₁₀-cycloalkyl radical, the carbon chain of which may optionally be interrupted, identically or differently, by one up to three oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s), or may be a group

—CH(XR¹)—CH₂(YR²)
in which
X and Y may each independently be oxygen, sulfur or —NR³—,
R¹ and R² may each independently be hydrogen or a branched or unbranched C₁-C₁₀-alkyl radical or C₄-C₁₀-cycloalkyl radical, the carbon chain of which may optionally be interrupted, identically or differently, by one or with up to three oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s),
R³ may be hydrogen or a C₁-C₆-alkyl radical which may be substituted by one or more hydroxyl group(s), thiol group(s) or amino group(s) and/or halogen atom(s),
R⁴ may be hydrogen or a C₁-C₆-alkyl radical which may be substituted by one or more hydroxyl group(s), thiol group(s) or amino group(s) and/or halogen atom(s),
   for purifying or for isolating biomolecules.

The present invention further preferably relates to the use of matrix materials of the general formula I

T—(C═O)O—B-D (I)
in which
T may be a polymer or copolymer comprising units of acrylic acid and/or methacrylic acid and/or esters thereof
B may be a covalent bond or a methylene group
D may be a branched or unbranched C₂-C₈-alkyl radical or C₃-C₈-cycloalkyl radical, the carbon chain of which may optionally be interrupted, identically or differently, by one up to two oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s), or may be a group

—CH(XR²)—CH₂(YR²)
in which
X and Y may each independently be oxygen, sulfur or —NR³—,
R¹ and R² may each independently be hydrogen or a branched or unbranched C₁-C₆-alkyl radical or C₄-C₆-cycloalkyl radical, the carbon chain of which may optionally be interrupted, identically or differently, by one or with up to two oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s),
R³ may be hydrogen or a C₁-C₃-alkyl radical which may be substituted by one or more hydroxyl group(s), thiol group(s) or amino group(s) and/or halogen atom(s),
R⁴ may be hydrogen or a C₁-C₃-alkyl radical which may be substituted by one or more hydroxyl group(s), thiol group(s) or amino group(s) and/or halogen atom(s),
   for purifying or for isolating biomolecules.

The present invention especially prefers the use of matrix materials of the general formula I

T—(C═O)O—B-D (I)
in which
T may be a crosslinked polymer consisting of units of methacrylic esters
B may be a covalent bond or a methylene group
D may be —CH₂—CH(OH)—CH₂OH, —CH₂—C(CH₂OH)₃, —CH₂—CH₂—O—CH₂—CH₂—OH
   or a group

—CH(XR²)—CH₂(YR²)
in which
X and Y may each be oxygen,
R¹ and R² may each independently be hydrogen, —CH(OH)—CH₂OH, —CH₂—CH(OH)—CH₂OH, —CH₂—C(CH₂OH)₃, —CH₂—CH₂—O—CH₂—CH₂—OH,
   for purifying or for isolating biomolecules.

The present invention most especially prefers the use of matrix materials of the general formula I

T-(C═O)O—B-D (I)
in which
T may be an ethylene glycol dimethacrylate-crosslinked polymer consisting of units of preferably one or more methacrylic ester(s),
B may be a covalent bond or a methylene group,
D may be —CH₂—CH₂OH, —CH₂—CH(OH)—CH₂OH, —CH₂—C(CH₂—OH)₃, —CH₂—CH₂—O—CH₂—CH₂—OH
   or a —CH(XR¹)—CH₂(YR²) group
   in which
   X and Y may be oxygen,
   R¹ and R² may each independently be hydrogen, —CH(OH)—CH₂OH, —CH₂—CH(OH)—CH₂OH, —CH₂—C(CH₂OH)₃, —CH₂—CH₂—O—CH₂—CH₂—OH,
      for purifying or for isolating biomolecules.

The present invention relates especially to the above-described uses of the inventive matrices for purification of biomolecules, wherein the biomolecule is one or more species of nucleic acid(s).

The present invention additionally relates to a process for isolating biomolecules with one of the inventive matrices, which comprises the following steps:
(a) providing the matrix;
(b) combining the matrix with a mixture comprising the biomolecule to be isolated or comprising preferably the nucleic acid to be isolated and at least one impurity;
(c) incubating the matrix and the mixture, in the course of which the biomolecule or preferably the desired nucleic acid is immobilized on the matrix;
(d) separating the matrix with the biomolecule or with the immobilized nucleic acid(s) from the mixture;
(e) combining the matrix with the biomolecule or with the immobilized nucleic acid(s) with an elution solution in, which desorbs the desired biomolecule(s) from the matrix.

The present invention relates especially to a method for isolating biomolecules with one of the inventive matrices, wherein the biomolecule is one or more species of nucleic acid(s).

The present invention additionally relates to nucleic acids which are bound to the inventive matrix material in the form of a complex.

The present invention additionally relates to a microfluidic system comprising a matrix material and to the use of the inventive matrix material in or in conjunction with a microfluidic system.

1. A matrix material of the general formula I
T-(C═O)O—B-D (I),
in which
T may be a polymer or copolymer comprising units of acrylic acid and/or derivatives thereof
B may be a covalent bond or an alkylene group having 1 to 6 carbon atoms
D may be a branched or unbranched C₂-C₂₀-alkyl radical or C₃-C₁₂-cycloalkyl radical, the carbon chain of which may optionally be interrupted, identically or differently, by one up to 10 oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s), or may be a group
—CH(XR¹)—CH₂(YR²)
in which
X and Y may each independently be oxygen, sulfur or —NR³—
and
R¹ and R² may each independently be hydrogen or a branched or unbranched C₁-C₂₀-alkyl radical or C₃-C₁₂-cycloalkyl radical, the carbon chain of which may optionally be interrupted, identically or differently, by one up to 10 oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s),
R³ may be hydrogen or a C₁-C₆-alkyl radical which may be substituted by one or more hydroxyl group(s), thiol group(s) or amino group(s) and/or halogen atom(s),
R⁴ may be hydrogen or a C₁-C₆-alkyl radical which may be substituted by one or more hydroxyl group(s), thiol group(s) or amino group(s) and/or halogen atom(s),
where, in the case that the support consists of a homopolymer of methacrylic acid, D must not be defined as —CH₂—CH₂OH.

2. The matrix material as claimed in claim 1, wherein
T may be a polymer or copolymer comprising units of acrylic acid and/or methacrylic acid and/or amides thereof and/or esters thereof
B may be a covalent bond or an alkylene group having 1 to 3 carbon atoms
D may be a branched or unbranched C₂-C₁₀-alkyl radical or C₃-C₁₀-cycloalkyl radical, the carbon chain of which may optionally be interrupted, identically or differently, by one up to three oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s), or may be a group
—CH(XR¹)—CH₂(YR²)
in which
X and Y may each independently be oxygen, sulfur or —NR³—,
R¹ and R² may each independently be hydrogen or a branched or unbranched C₁-C₁₀-alkyl radical or C₄-C₁₀-cycloalkyl radical, the carbon chain of which may optionally be interrupted, identically or differently, by one or with up to three oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s),
R³ may be hydrogen or a C₁-C₆-alkyl radical which may be substituted by one or more hydroxyl group(s), thiol group(s) or amino group(s) and/or halogen atom(s),
R⁴ may be hydrogen or a C₁-C₆-alkyl radical which may be substituted by one or more hydroxyl group(s), thiol group(s) or amino group(s) and/or halogen atom(s),
where, in the case that the support consists of a homopolymer of methacrylic acid, D must not be defined as —CH₂—CH₂OH.

3. The matrix material as claimed in claim 2, wherein
T may be a polymer or copolymer comprising units of acrylic acid and/or methacrylic acid and/or esters thereof
B may be a covalent bond or a methylene group
D may be a branched or unbranched C₂-C₈-alkyl radical or C₃-C₈-cycloalkyl radical, the carbon chain of which may optionally be interrupted, identically or differently, by one up to two oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s), or may be a group
—CH(XR¹)—CH₂(YR²)
in which
X and Y may each independently be oxygen, sulfur or —NR³—,
R¹ and R² may each independently be hydrogen or a branched or unbranched C₁-C₆-alkyl radical or C₄-C₆-cycloalkyl radical, the carbon chain of which may optionally be interrupted, identically or differently, by one or with up to two oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s),
R³ may be hydrogen or a C₁-C₃-alkyl radical which may be substituted by one or more hydroxyl group(s), thiol group(s) or amino group(s) and/or halogen atom(s),
R⁴ may be hydrogen or a C₁-C₃-alkyl radical which may be substituted by one or more hydroxyl group(s), thiol group(s) or amino group(s) and/or halogen atom(s),
where, in the case that the support consists of a homopolymer of methacrylic acid, D must not be defined as —CH₂—CH₂OH.

4. The matrix material as claimed in claim 3, wherein
T may be a crosslinked polymer consisting of units of methacrylic esters
B may be a covalent bond or a methylene group
D may be —CH₂—CH(OH)—CH₂OH, —CH₂—C(CH₂OH)₃, —CH₂—CH₂—O—CH₂—CH₂—OH
or a group
—CH(XR¹)—CH₂(YR²)
in which
X and Y may be oxygen,
R¹ and R² may each independently be hydrogen, —CH(OH)—CH₂OH, —CH₂—CH(OH)—CH₂OH, —CH₂—C(CH₂OH)₃, —CH₂—CH₂—O—CH₂—CH₂—OH.

5. The matrix material as claimed in claim 4, wherein
T may be an ethylene glycol dimethacrylate-crosslinked polymer consisting of units of preferably one or more methacrylic ester(s),
B may be a covalent bond or a methylene group,
D may be —CH₂—CH₂OH, —CH₂—CH(OH)—CH₂OH, —CH₂—C(CH₂—OH)₃, —CH₂—CH₂—O—CH₂—CH₂—OH
or a group —CH(XR¹)—CH₂(YR²)
in which
X and Y may be oxygen,
R¹ and R² may each independently be hydrogen, —CH(OH)—CH₂OH, —CH₂—CH(OH)—CH₂OH, —CH₂—C(CH₂OH)₃, —CH₂—CH₂—O—CH₂—CH₂—OH.

6. A matrix comprising a matrix material as claimed in claim 1, wherein the matrix material is bonded to a material which has a permanent or temporary magnetic moment.

7. The matrix comprising a matrix material as claimed in claim 6, wherein the material having a permanent or temporary magnetic moment is ferri- or ferromagnetic or para- or superparamagnetic.

8. A matrix comprising a matrix material as claimed in claim 1, wherein it is in the form of beads.

9. A matrix comprising a matrix material as claimed in claim 1, wherein the matrix is in the form of a composite.

10. A process for producing matrix materials as claimed in claim 1, wherein
a) the monomers which build up the support material and which bear the epoxide partial structure are polymerized, optionally in the presence of a material which possesses a permanent or temporary magnetic moment, and
b) the resulting polymers are subjected to a hydrolysis, alcoholysis, aminolysis, or are reacted with a thiol, and
c) the derivatized polymers are isolated.

11. A process for producing matrix materials as claimed in claim 1, wherein
a) the monomers which build up the support material and which bear the ester partial structure are polymerized, optionally in the presence of a crosslinker and/or of a material which possesses a permanent or temporary magnetic moment, and
b) the resulting polymers are subjected to a transesterification with an alcohol and
c) the derivatized polymers are isolated.

12. The use of a matrix material of the general formula I
T-(C═O)O—B-D (I),
in which
T may be a polymer or copolymer comprising units of acrylic acid and/or derivatives thereof
B may be a covalent bond or an alkylene group having 1 to 6 carbon atoms
D may be a branched or unbranched C₂-C₂₀-alkyl radical or C₃-C₁₂-cycloalkyl radical, the carbon chain of which may optionally be interrupted, identically or differently, by one up to 10 oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s), or may be a group
—CH(XR¹)—CH₂(YR²)
in which
X and Y may each independently be oxygen, sulfur or —NR³—
and
R¹ and R² may each independently be hydrogen or a branched or unbranched C₁-C₂₀-alkyl radical or C₃-C₁₂-cycloalkyl radical, the carbon chain of which may optionally be interrupted, identically or differently, by one up to 10 oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s),
R³ may be hydrogen or a C₁-C₆-alkyl radical which may be substituted by one or more hydroxyl group(s), thiol group(s) or amino group(s) and/or halogen atom(s),
R⁴ may be hydrogen or a C₁-C₆-alkyl radical which may be substituted by one or more hydroxyl group(s), thiol group(s) or amino group(s) and/or halogen atom(s),
for purifying or for isolating biomolecules.

13. The use as claimed in claim 12, wherein
T may be a polymer or copolymer comprising units of acrylic acid and/or methacrylic acid and/or amides thereof and/or esters thereof
B may be a covalent bond or an alkylene group having 1 to 3 carbon atoms
D may be a branched or unbranched C₂-C₁₀-alkyl radical or C₃-C₁₀-cycloalkyl radical, the carbon chain of which may optionally be interrupted, identically or differently, by one up to three oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s), or may be a group
—CH(XR¹)—CH₂(YR²)
in which
X and Y may each independently be oxygen, sulfur or —NR³—
R¹ and R² may each independently be hydrogen or a branched or unbranched C₁-C₁₀-alkyl radical or C₄-C₁₀-cycloalkyl radical, the carbon chain of which may optionally be interrupted, identically or differently, by one or with up to three oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s),
R³ may be hydrogen or a C₁-C₆-alkyl radical which may be substituted by one or more hydroxyl group(s), thiol group(s) or amino group(s) and/or halogen atom(s),
R⁴ may be hydrogen or a C₁-C₆-alkyl radical which may be substituted by one or more hydroxyl group(s), thiol group(s) or amino group(s) and/or halogen atom(s).

14. The use as claimed in claim 13, wherein
T may be a polymer or copolymer comprising units of acrylic acid and/or methacrylic acid and/or esters thereof
B may be a covalent bond or a methylene group
D may be a branched or unbranched C₂-C₈-alkyl radical or C₃-C₈-cycloalkyl radical, the carbon chain of which may optionally be interrupted, identically or differently, by one up to two oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s), or may be a group
—CH(XR¹)—CH₂(YR²)
in which
X and Y may each independently be oxygen, sulfur or —NR³—
R¹ and R² may each independently be hydrogen or a branched or unbranched C₁-C₆-alkyl radical or C₄-C₆-cycloalkyl radical, the carbon chain of which may optionally be interrupted, identically or differently, by one or with up to two oxygen, sulfur or bivalent amino group(s) —NR⁴—, and which may be substituted by one or more hydroxyl group(s), thiol group(s) or amino group(s), aldehyde group(s), carboxamide group(s), sulfonamide group(s) and/or halogen atom(s),
R³ may be hydrogen or a C₁-C₃-alkyl radical which may be substituted by one or more hydroxyl group(s), thiol group(s) or amino group(s) and/or halogen atom(s),
R⁴ may be hydrogen or a C₁-C₃-alkyl radical which may be substituted by one or more hydroxyl group(s), thiol group(s) or amino group(s) and/or halogen atom(s).

15. The use as claimed in claim 14, wherein
T may be a crosslinked polymer consisting of units of methacrylic esters
B may be a covalent bond or a methylene group
D may be —CH₂—CH(OH)—CH₂OH, —CH₂—C(CH₂OH)₃, —CH₂—CH₂—O—CH₂—CH₂—OH
or a group
CH(XR¹)—CH₂(YR²)
in which
X and Y may be oxygen,
R¹ and R² each independently be hydrogen, —CH(OH)—CH₂OH, —CH₂—CH(OH)—CH₂OH, —CH₂—C(CH₂OH)₃, —CH₂—CH₂—O—CH₂—CH₂—OH.

16. The use as claimed in claim 15, wherein
T may be an ethylene glycol dimethacrylate-crosslinked polymer consisting of units of preferably one or more methacrylic ester(s),
B may be a covalent bond or a methylene group,
D may be —CH₂—CH₂OH, —CH₂—CH(OH)—CH₂OH, —CH₂—C(CH₂—OH)₃, —CH₂—CH₂—O—CH₂—CH₂—OH
or a —CH(XR¹)—CH₂(YR²) group
in which
X and Y may be oxygen,
R¹ and R² may each independently be hydrogen, —CH(OH)—CH₂OH, —CH₂—CH(OH)—CH₂OH, —CH₂—C(CH₂OH)₃, —CH₂—CH₂—O—CH₂—CH₂—OH.

17. The use as claimed in claim 12, wherein the biomolecule comprises one or more species of nucleic acid(s).

18. A method for isolating biomolecules having a matrix as claimed in claim 1, comprising the following steps:
(a) providing the matrix;
(b) combining the matrix with a mixture comprising the biomolecule to be isolated and at least one impurity;
(c) incubating the matrix and the mixture, in the course of which the biomolecule is immobilized on the matrix;
(d) separating the matrix with the biomolecule, or from the mixture;
(e) combining the matrix with the biomolecule or with an elution solution in, which desorbs the desired biomolecule(s) from the matrix.

19. The method as claimed in claim 18, wherein the biomolecule comprises one or more species of nucleic acid(s).

20. A nucleic acid, wherein it is bound in the form of a complex with the matrix material as claimed in claim 1.

21. A microfluidic system comprising a matrix material as claimed in claim 1.
